(19) United States
(12) Patent Application Publication
(10) Pub. No.: US 2004/0136944 A1
(43) Pub. Date: Jul. 15, 2004

(54) Shaving systems

(75) Inventors: Susan Rae Anderson, Pensacola, FL (US); Thomas H. Taylor, Guilford, CT (US)

Correspondence Address: Richard R. Michaud, McCormick, Paulding & Huber LLP, CityPlace II, 185 Asylum Street, Hartford, CT, 06103, US

(73) Assignee: Eveready Battery Company, Inc., Westlake, OH

(21) Appl. No.: 10/692,247

(22) Filed: Oct. 23, 2003

Related U.S. Application Data
(60) Provisional application No. 60/424,361, filed on Nov. 6, 2002.

(51) Int. Cl. A61K 7/15

(52) U.S. Cl. 424/73

(57) ABSTRACT

Shaving systems are disclosed which comprise a blade member and a skin engaging portion in proximity to the blade member, wherein the skin engaging portion includes a chelating agent capable of reducing the amount of insoluble metal salts in an aqueous liquid containing the insoluble metal salts, e.g., hard water. The chelating agent may be incorporated into a shaving aid strip or into another component of the shaving system, e.g., the leading edge or trailing edge of the shaving head. Alternatively, the chelating agent may be provided as a coating, optionally with water-sensitive polymers, to the skin engaging portion of the shaving system.

### CROSS-REFERENCE TO RELATED APPLICATIONS

This application is entitled to the benefit of and incorporates by reference essential subject matter disclosed in Provisional Patent Application No. 60/424,361 filed on Nov. 6, 2002.

### FIELD OF THE INVENTION

The present invention generally relates to shaving systems, i.e., razors, and more specifically relates to shaving systems which contain chelating agents capable of reducing the amount of insoluble metal salts in aqueous liquids containing insoluble metal salts e.g., water used for shaving.

### BACKGROUND OF THE INVENTION

In shaving systems of the wet shave type, factors such as the frictional drag of the razor across the skin, the force needed to sever hairs, and irritation of preexisting skin conditions can create shaving discomfort. It is known to incorporate a shaving aid into a razor by mounting a shaving aid strip to the razor for the purpose of alleviating shaving discomfort. For example, U.S. Pat. No. 5,113,585, issued May 19, 1992, discloses a shaving aid strip including a water-insoluble matrix material, a water-soluble shaving aid, and a low molecular weight release-enhancing agent. When exposed to water during shaving, the water-soluble shaving aid leaches from the shaving aid strip onto the skin. The release-enhancing agent also dissolves in the water and improves the release of the water-soluble shaving aid from the shaving aid strip.

Water used for shaving often contains varying degrees of insoluble metal salts, which may be present, for example, as calcium and magnesium carbonates, bicarbonates, sulfates, or chlorides. Such salts are typically formed as a result of water contacting rocky substances and soils, e.g., well water. Water containing about 100 micrograms per liter (“μg/L”) or more of salt, e.g., calcium carbonate, is usually considered to be “hard water”. Depending upon the skin sensitivity of the person shaving and the amount of insoluble metal salts in the water, the salts can cause additional skin irritation above and beyond that caused by the act of shaving itself. In addition, high concentrations of insoluble metal salts can lead to the formation of deposits that can appear as scaling and staining on the blades of the razor. The buildup of such deposits can lead to unsightly appearance of the blades and increased shaving discomfort and a decrease in the useful life of the shaving system.

Accordingly, improved shaving systems are desired which can accommodate the use of aqueous liquids containing insoluble metal salts. Desirably, such improved shaving systems would provide less shaving discomfort and skin irritation than provided by currently available shaving systems.

### SUMMARY OF THE INVENTION

In accordance with the present invention, improved shaving systems are provided which contain chelating agents capable of reducing the amount of insoluble metal salts in aqueous liquids containing the insoluble metal salts, e.g. hard water. The chelating agent can be present in a shaving aid strip or in another component of the shaving system such as, for example, the shaving head. In a preferred aspect of the invention, the chelating agent can be provided by coextruding the chelating agent with thermoplastic polymers used in the shaving aid strip or other components of the shaving system, or by delivering the chelating agent in a liquid form, i.e., from a reservoir or cavity in the shaving head. Alternatively, in another preferred aspect of the invention, the chelating agent can be provided as a coating such as, for example, as a component in a lubricious polymer coating applied to the skin engaging portion of the shaving head.

By virtue of the present invention, it is now possible to provide shaving systems which can accommodate insoluble metal salts in the shaving water and provide less shaving discomfort, particularly for individuals with sensitive skin. Additionally, the amount of scale buildup on the blades of shaving systems can be reduced using the shaving systems of the present invention. Quite advantageously, due to the avoidance of scale formation and buildup, the useful life of the shaving systems of the present invention can be increased.

### BRIEF DESCRIPTION OF THE DRAWING

FIG. 1 is a perspective view of a shaving system in accordance with the present invention.

### DETAILED DESCRIPTION OF THE PREFERRED EMBODIMENT

The metal ions that form the insoluble metal salts which can be removed by the shaving systems of the present invention are not critical and include those typically found as dissolved carbonates and other salts in potable water, e.g., household water supplies. Typical of such ions include calcium, magnesium, iron, copper, zinc and mixtures thereof. The shaving systems of the present invention can be used to remove ions of other Group II and transition metals, as noted in the Periodic Table of Elements “General Chemistry Principles and Modern Applications”, Seventh Edition, Petrucci, R. H. and Harwood, W. S.; Prentice-Hall, Inc., inside cover, 1997. The concentration of the insoluble metal salts contained in the aqueous liquids is not critical to the present invention and usually is from about 1 to 300 μg/L and more typically from about 10 to 150 μg/L, based on the total volume of the aqueous liquid.

The chelating agents suitable for use in accordance with the present invention include those capable of reducing the amount of insoluble metal salts in the aqueous liquid. The chelating agents include compounds or polymers having at least one functional group capable of forming a chelate with a metal ion of the insoluble metal salt. As used herein, the term “chelate” includes coordination compounds in which a metal ion such as calcium or magnesium is attached by coordinate links to one or more nonmetal atoms in the same molecule (also referred to in the art as ligands). One or more chelating agents can be used in the shaving systems of the present invention.

The particular chelating agents suitable for use in accordance with the present invention are not critical. However, the chelating agents selected should be safe for human use. They may be polymeric or nonpolymeric. When in polymeric form, the chelating agents will typically have a molecular weight from about 300 to 10,000,000 grams per gram mole (“g/mol”) with a range of 70,000 to 3,000,000 g/mol being more common. As used herein the term “molecular weight” means weight average molecular weight. Techniques for determining the weight average molecular weight of polymers are known to those skilled in the art. One such technique is gel permeation chromatography. Preferably when in polymeric form, the chelating agents are compatible with other polymers used in the shaving system. That is, the chelating agent can be coextruded with other polymers used in the shaving system, e.g., polyethylene oxide or polystyrene, and form a reasonably homogenous blend. Quite advantageously, in accordance with the present invention, the chelating agent can be provided as a separate polymer, i.e., in addition to the other polymers used in the shaving system, or chelating functionality can be added to one of the other polymers used in the shaving system, e.g., the water-sensitive polymers or water-insoluble polymers (hereinafter described). For example, other polymers typically used in the shaving system, e.g., polymers used to provide lubricity or structural rigidity, can be derivitized to contain the chelating functionality. Alternatively, monomers containing the desired chelating functionality can be copolymerized with other monomers used in the shaving system to obtain the desired polymer.

Typical chelating agents suitable for use in accordance with the present invention may contain one or more of the following functional groups: sulfonates, phosphates, phosphines, phosphites, carboxylic acids, amines and silicates. Examples of chelating agents include ethylenediaminetetracetic acid (“EDTA”), nitritotriacetic acid, ethylene glycol-bis-(β-aminoetyl ether)-N,N-tetraacetic acid, (ethlenedioxy)-diethylene-dinitrilo-tetra-acetic acid, salicylaldoxime, quinolinol, diaminocyclohexanne-tetra-acetic acid, diethylene-triamino-penta-acetic acid, dimethylglyoxime, benzoin oxime, triethylenetramine, desferrioxamine calcium disodium EDTA, dipotassium EDTA, disodium EDTA, methyl cyclodextrin, pentenic acid, potassium citrate, sodium citrate, sodium gluconate, sodium metasilicate, tetrasodium EDTA, trisodium EDTA and poly(sodium 4-styrenesulfonate). Further examples of chelating agents are described, for example, in U.S. Pat. No. 5,487,884 issued Jan. 30, 1996, U.S. Pat. No. 5,741,945 issued Apr. 21, 1998, U.S. Pat. No. 5,801,139 issued Sep. 1, 1998, and the International Cosmetic Ingredient Dictionary and Handbook, Ninth Edition 2002, volume 4, page 2892 published by The Cosmetic, Toiletry, and Fragrance Association.

Preferred chelating agents for use in accordance with the present invention have functional groups selected from the group consisting of (—SOₓ^{-y}) (Z^{+w}), (—POₓ^{-y}) (Z^{+w}), (R—COO^{-y}) (Z^{+x}), where x is 2, 3, or 4, y is less than or equal to x, R is any alkyl, aryl or aryl alkyl group, Z is any cation(s) and w is 1, 2, 3 or 4. Preferred chelating agents include calcium disodium EDTA, dipotassium EDTA, disodium EDTA, methyl cyclodextrin, pentenic acid, potassium citrate, sodium citrate, sodium gluconate, sodium metasilicate, tetrasodium EDTA, trisodium EDTA, poly(sodium 4-styrenesulfonate). An especially preferred chelating agent is poly(sodium 4-styrenesulfonate).

Preferably, the chelating agents suitable for use and in accordance with the present invention are effective to reduce the amount of insoluble metal salts in the aqueous liquids by at least about 10 percent, more preferably by at least about 20 percent and most preferably by least about 30 percent. The amount of reduction in metal salt content can be determined by placing a shaving head, e.g. a razor cartridge, in 60 milliliters of the aqueous liquid containing the insoluble metal salt, allowing the shaving head to remain in the liquid until equilibrium is achieved, i.e. for approximately 24 hours, and analyzing the concentration of the metal ions in the aqueous liquid. An increase in the amount of metal ions in the liquid indicates a corresponding decrease in the amount of insoluble metal salt that dissociates to liberate the metal ion. By comparing the concentration of metal ions in the aqueous liquid after conducting the test with the concentration of metal ions in the aqueous liquid that does not contain the shaving head, e.g., at the beginning of the test, the amount of metal salt solubilized can be easily calculated. This test is further demonstrated in Example 3. As set forth in Example 3, for purposes of experimental convenience, it is preferred that an excess of insoluble metal salt be used to conduct the test to measure the reduction in salt content (increase in metal ion content). The amount of ions in the aqueous liquid can be determined by routine analytical techniques known to those skilled in the art, e.g. Inductively Coupled Plasma (“ICP”) or atomic absorption.

Further details concerning the selection of suitable chelating agents can be determined by those skilled in the art.

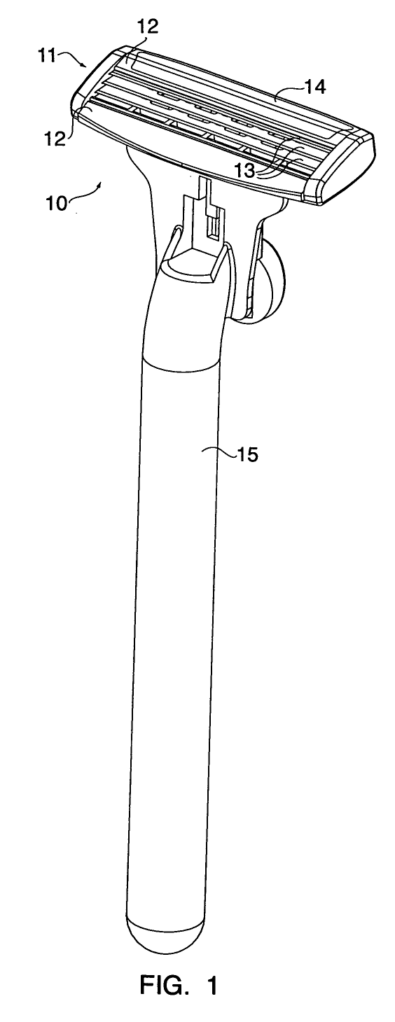

The invention is further described with reference to FIG. 1 which is not intended to limit the scope of the claims which follow.

Referring to FIG. 1, a shaving system 10 is provided which includes a shaving head 11 including a skin engaging portion 12 in which three blade members 13 are fixed in proximity to the skin engaging portion 13. Shaving head 11 further contains a solid polymeric shaving aid strip 14 mounted near the trailing edge (also referred to in the art as the cap) of the shaving head. Alternatively, the shaving aid strip may be placed at the leading edge (also referred to in the art as the guard portion) of the shaving head or at both positions on the shaving head. Shaving head 11 is generally affixed to a handle 15 for use in shaving. The shaving head may be designed to be removeably affixed to the handle in the case of shaving systems in which the handle is reused and the shaving head is disposed of after one or more uses. Alternatively, the shaving head may be permanently affixed to the handle (or integral with the handle) in the case of disposable razors in which the handle and cartridge are disposed of together as a unit after one or more uses. As used herein, the term “shaving system” is intended to include both types of shaving heads.

The shaving system components, e.g., shaving head 11 or shaving aid strip 14, are typically comprised of one or more thermoplastic water-insoluble polymers which provide structural integrity to the shaving system. The particular water-insoluble polymers are not critical to the present invention. Examples of suitable water-insoluble polymers for use in the shaving systems of the present invention are selected from the group consisting of polyethylene, polypropylene, polystyrene, butadiene-styrene copolymer, polyacetal, acrylonitrile-butadiene-styrene copolymer, ethylene vinyl acetate copolymer, polyurethanes and mixtures thereof. Typically, when water-insoluble polymers are present in the various shaving system components, they are present in an amount of at least 5 weight percent, preferably from about 10 to 100 weight percent, and more preferably from about 20 to 50 weight percent, based upon the total weight of the shaving system component. Suitable water-insoluble polymers are commercially available. Further details concerning the selection and amounts of the water-insoluble polymers are known to those skilled in the art.

The water-insoluble polymers may be blended with water-sensitive polymers in order to achieve the desired lubricity properties, e.g., in the shaving aid strip. As used herein, the term “water-sensitive” means water-soluble or water-swellable. The term “water-soluble” means that at least 0.2 weight percent, preferably at least 0.5 weight percent, of the polymer is soluble in distilled water (pH=7.0) at 25° C. and 1 atmosphere pressure; and the term “water-swellable” means that the weight of the polymer increases by at least 2 percent, preferably at least 5 weight percent, after being immersed in water at room temperature, e.g., 25° C., for 1 hour.

The particular water-sensitive polymers are not critical to the present invention. Examples of water-sensitive polymers suitable for use in accordance with the present invention polymers include, but are not limited to, those selected from the group consisting of polyvinyl compounds, polysaccharides, polyurethanes, polyacrylates, polyacrylamides, polyalkylene oxides, polysulfones, polylactones, polylactams and copolymers, complexes, mixtures, and derivatives thereof. Other preferred water-sensitive polymers for use in accordance with the present invention include, but are not limited to, those selected from the group consisting of N-vinylpyrrolidone-hydroxyethyl acrylate copolymers, carboxymethylcellulose, hydroxyethyl cellulose, polyacrylamide, polyhydroxyethyl-acrylate, cationically-modified hydroxyethylcellulose, polyacrylic acid, polyethylene oxides, and complexes, mixtures, and derivatives thereof.

The water-sensitive polymers suitable for use in accordance with the present invention can be nonionic, cationic, anionic or amphoteric. Typically, the weight average molecular weight of the water-sensitive polymers is from about 100,000 to 10,000,000 g/mol, preferably from about 200,000 to 8,000,000 g/mol, and, more preferably, from about 300,000 to 5,000,000 g/mol. In general, details concerning the preparation and selection of water-sensitive polymers suitable for use in accordance with the present invention are known to those skilled in the art. Such hydrophilic polymers are readily commercially available from a variety of sources.

Especially preferred water-sensitive soluble polymers are polyethylene oxides. The more preferred polyethylene oxides generally are known as POLYOX™ Water Soluble Resins (available from The Dow Chemical Company, Midland, Mich.) or ALKOX™ Resins (available from Meisei Chemical Works, Kyoto, Japan). It is often preferred to use a blend of polyethylene oxides. A typical a blend has at least one polyethylene oxide having a weight average molecular weight in the range of 100,000 to 500,000 g/mol and at least one polyethylene oxide having a weight average molecular weight in the range of 3,000,000 to 8,000,000 g/mol. An especially preferred polyethylene oxide comprises about 40 percent to 100 percent by weight of polyethylene oxide having a weight average molecular weight of about 5,000,000 g/mol (e.g. POLYOX™ WSR COAGULANT) and about 60 percent to 0 percent by weight of polyethylene oxide having a weight average molecular weight of about 300,000 g/mol (e.g. POLYOX™ WSR N-750).

Typically, when water-sensitive polymers are utilized in the various shaving system components, they are present in an amount of from about 1 to 100 percent, preferably from about 10 to 90 weight percent and more preferably from about 50 to 80 weight percent, based on the total weight of shaving system component. Suitable water-sensitive polymers are commercially available. Further details concerning the selection and amount of the water-sensitive polymers, including blends of water-insoluble polymers and water-sensitive polymers, are known to those skilled in the art. For example, molded polymer blends comprising a polyolefin, e.g. polyethylene, a polyalkylene oxide, e.g., polyethylene oxide, and a polylactone e.g., polycaprolactone, are disclosed in U.S. Pat. No. 5,589,545 issued Dec. 31, 1996.

Shaving system components of the present invention may be fabricated by any appropriate method, including, for example, injection molding and extrusion. For example, in the manufacture of the shaving aid strip, all of the components of the strip may be blended prior to molding or extrusion. For best results, it is usually preferred that the components are dry.

The blended components may be extruded, for example, through a Haake System 90, ¾ inch diameter extruder with a barrel pressure of about 1000-2000 pounds per square inch gauge (“psi”), a rotor speed of about 10 to 50 revolutions per minute (“rpm”) and a temperature of about 150° C. to 185° C. and a die temperature of about 170° C. to 185° C. Alternatively, a 11/4 inch single screw extruder may be employed with a processing temperature of 175° C. to 200° C., preferably 185° C. to 190° C., a screw speed of 20 to 50 rpm, preferably 25 to 35 rpm, and an extrusion pressure of 1800 to 5000 psi, preferably 2000 to 3500 psi. The extruded strip is typically air cooled to about 25° C. The powder mix of ingredients can be molded in either a single material molding machine or multi-material molding machine, which may be single cavity or multi-cavity, optionally equipped with a hot runner system. The process temperature can be from 165° C. to 250° C., preferably from 180° C. to 225° C. The injection pressure should be sufficient to fill the part completely without flashing. Depending on the cavity size, configuration and quantity, the injection pressure can range from 300 to 2500 psi or greater. The cycle time is typically from about 3 to 30 seconds, with the optimum generally being about 6 to 15 seconds.

Alternatively, the ingredients may be supplied by two separate melting/pumping (plastics extruders), each comprising of a heated barrel, a pumping screw, a motor drive for that screw and a control system for the entire system. The ingredients are typically fed in powder form into their respective extruders (e.g., single screw type manufactured by Davis Standard Corporation, Pawcatuck, Conn.). The extruders can operate at the same or different speeds and the same or different temperatures. The barrel temperature for each extruder can be ramped, for example, in three zones from 160° C. to 190° C.; a fourth heater at the die/barrel connection can also be set to about 190° C., and a fifth heater at the die can range from 190° C. to 200° C. Then, the molten streams of the components are brought together to form the shaving aid strip. The final dimensions are achieved using a series of forming rollers as the extrudate is cooled. The composite is typically extruded at a rate of about 50 feet per minute. The combined molten materials are desirably drawn from the die head into the sizing/cooling device at a constant speed such that its cross section is constant. Under a bath of cool dry air, the molten material is cooled until no longer pliable. Once cooled, the shaving system component can be further processed as desired.

In a preferred aspect of the invention, the chelating agent is provided to an internal portion of a shaving system component, e.g., the guard or cap of the shaving head or the shaving aid strip, by blending and coextruding the chelating agent with the polymers used to make the shaving system component. For example, in the manufacture of a shaving head, poly(sodium 4-styrene sulfonate) may be blended with polystyrene and extruded to form the housing of the shaving head. When the chelating agent is provided to an internal portion of the shaving system component, it is typically provided in an amount of from about 0.1 to 10 weight percent, preferably from about 0.5 to 6 weight percent, more preferably from about 1 to 4 weight percent, based on the total weight of the shaving system component. Alternatively, the skin engaging portion of the shaving head may be provided with a reservoir or cavity containing a liquid which comprises the chelating agent and optionally other conventional shaving aid ingredients (further described below). The liquid may be delivered through holes in the shaving head such as disclosed in U.S. Pat. No. 6,298,558, issued Oct. 9, 2001. In such cases where the chelating agent is subject to extrusion or other molding conditions, it is preferred that the chelating agent has a decomposition temperature which is higher than the processing temperature. Preferably, the chelating agents of the present invention have a decomposition temperature of at least 150° C., more preferably at least about 200° C. and most preferably at least about 250° C. Thus, in this aspect of the invention, there is provided a shaving system component comprising a solid polymeric element having an outer surface and an internal portion, the polymeric element comprising: (i) at least one of a water-sensitive polymer and (ii) a water-insoluble polymer; and the chelating agent.

In an especially preferred aspect of the invention, the chelating agent is provided in conjunction with a shaving aid strip. Typically, the shaving aid strip is fixed in an opening in the rear of the skin engaging portion of the shaving head and is comprised of one or more water-sensitive polymers that are released by the strip during shaving to improve shave attributes. While shaving aid strip 14 is shown in FIG. 1 at the trailing edge of shaving head 11, the shaving aid strip may be located at any skin engaging portion of the shaving head, e.g., at the leading edge of the skin engaging portion, and may be fabricated in any size or shape deemed appropriate. Typically the shaving aid strip will comprise from about 0 to 40 weight percent, preferably from about 10 to 35 weight percent of one or more water-insoluble polymers, from about 50 to 99.9 weight percent, preferably from about 59 to 89.5 weight percent of one or more water-sensitive polymers, and from about 0.1 to 10 weight percent, preferably from about 0.5 to 6 weight percent and more preferably from about 1 to 4 weight percent of one or more chelating agents, based on the total weight of the shaving aid strip.

The shaving aid strip may also contain other conventional shaving aid ingredients, such as low molecular weight water-sensitive release enhancing agents such as a polyethylene glycol having a weight average molecular weight of about 5,000 g/mol or less (e.g., 1-10% by weight), water-sensitive release enhancing agents such as cross-linked polyacrylics (e.g., 2-7% by weight), colorants, antioxidants, preservatives, botanical oils, vitamin E, aloe, cooling agents, essential oils, beard softeners, astringents, medicinal agents, etc. Portions that contain a colorant can be designed to release the colorant (e.g., by leaching or abrasion), and thereby cause the strip to change color during shaving. Typically, such portions may contain, for example, between about 0.1% and about 5.0% (preferably between about 0.5% and 3%) colorant by weight. Further details of shaving aid strips are known to those skilled in the art. See, for example, U.S. Pat. No.6,301,785, issued, Oct. 16, 2001, U.S. Pat. No. 6,298,558, issued Oct. 9, 2001, and U.S. Pat. No. 6,185,822, issued Feb. 13, 2001.

In another aspect of the invention, the chelating agent is provided as a coating to at least a portion of the skin engaging portion of the shaving system. Advantageously, the chelating agent can be included with water-sensitive polymers to provide a lubricious-when-wet coating to the shaving device. As used herein the term “lubricious-when-wet” refers to a reduction in the coefficient of friction of an article upon exposure to water. Details concerning the measurement of an article's coefficient of friction are known to those skilled in the art. Examples of suitable lubricious-when-wet coatings are disclosed, for example, in U.S. Pat. No. 4,875,287 issued Oct. 24, 1989; U.S. Pat No. 5,620,738 issued Apr. 15, 1997 and U.S. Pat. No. 5,091,205 issued Feb. 25, 1992. When the chelating agent is provided to the shaving system as a coating, it is typically present in an amount of from about 0.1 to 10.0 weight percent, preferably from about 0.5 to 6.0 weight percent, with the most preferred range of about 1.0 weight percent to 4.0 weight percent, based on the total weight of solids, i.e. polymers plus the chelating agent, in the coating liquid. When the chelating agent is applied in the form of a coating, the decomposition temperature of the chelating agent is not significant, as it is in the case when the chelating agent is coextruded with other polymers which comprise the shaving system component.

The following examples are provided for illustrative purposes and are not intended to limit the scope of the claims which follow. The experiments demonstrate the ability of a chelating agent (PSSNa) to complex with metal ions of an insoluble metal salt (calcium carbonate) of an aqueous liquid containing the salt in accordance with the present invention. Those skilled in the art can select other chelating agents for the particular insoluble salts to be removed by following the examples set forth below. The following chemical products were used in the Examples.

| Name | Description |
|---|---|
| PSSNa | poly(sodium 4-styrenesulfonate), having a molecular |
| | weight of about 70,000 g/mol, available from |
| | National Starch & Chemical Company, Berkeley, CA. |
| MIPS | medium impact polystyrene, specific gravity of about 1.06, available from |
| | PolyOne Corporation, Cleveland, OH. |
| POLYOX ™ | Coagulant polyethylene oxide having an average molecular weight of |
| | about 5,000,000, available from The Dow Chemical |
| | Company, Midland, MI. |
| POLYOX ™ WSR N-750 | polyethylene oxide having an average molecular weight |
| | of about 300,000, available from The Dow Chemical |
| | Company, Midland, MI. |
| PEG | polyethylene glycol having an average molecular weight of about 3000, |
| | available from The Dow Chemical Company, Midland, |
| | MI. |
| PP | polypropylene having an average specific gravity of about 0.91, available |
| | from PolyOne Corporation, Cleveland, OH. |
| Aloe | a natural plant extract with a variety of components with molecular weights |
| | ranging from 194 to 540. |

### EXAMPLE 1

### Dissolution of Insoluble Salt

This experiment demonstrates the ability of a chelating agent to complex with metal ions in water. In twelve separate beakers, one gram of CaCO₃ was added to each beaker followed by the addition of 60 milliliters (“ml”) of deionized water. This created an excess of insoluble metal salt. The beakers were separated into four groups of three beakers. In the first group, no chelating agent was added. In the second group 1.000 g of PSSNa was added to each of the three beakers. In the third group 0.5000 g of PSSNa was added to each of the three beakers. In the fourth group 0.2500 g of PSSNa was added to each of the three beakers. The beakers were allowed to equilibrate for 24 hours. Then, the contents of each beaker were filtered and the supernatant was analyzed by ICP for calcium content. Table 1, below, sets forth the results of the analysis.

TABLE 1

| Group | Beaker | Calcium Content, μg/L |
|---|---|---|
| I | 1 | 17.0 |
| | 2 | 15.5 |
| | 3 | 18.2 |
| II | 4 | 38.7 |
| | 5 | 33.3 |
| | 6 | 34.7 |
| III | 7 | 35.0 |
| | 8 | 36.2 |
| | 9 | 32.1 |
| IV | 10 | 39.0 |
| | 11 | 47.3 |
| | 12 | 37.3 |

The beakers in which PSSNa was added showed an increase in Ca⁺² by approximately 2 times regardless of the amount of PSSNa added (see Table 2). Since CaCO₃ was in excess in all the beakers, any increase in Ca⁺² was attributed to the effect of the PSSNa binding up the soluble Ca⁺² and then some insoluble CaCO₃ going into solution to restore equilibrium. This experiment demonstrates that the PSSNa does indeed bind with the calcium in water. Without being bound to any particular theory, the equilibrium equation is believed to be as follows:

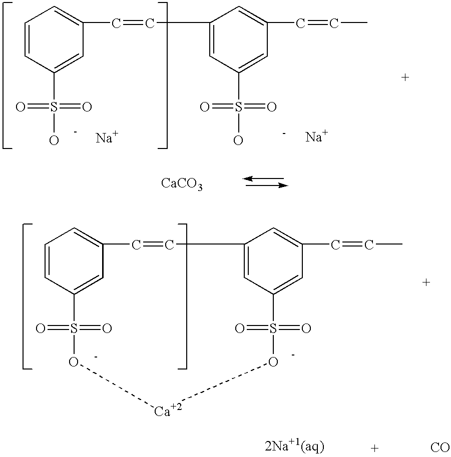

### EXAMPLE 2

### Manufacture of Shaving Head

This example demonstrates the manufacture of a shaving head containing a shaving aid strip in accordance with the present invention. Shaving heads containing shaving aid strips comprising the formulations set for the in Table 2 were manufactured using conventional equipment and an injection molding process as described below. The PP and blades were sequentially molded at an elevated temperature and pressure. The cartridge was then sent to the next station where the dry, blended components of the formulas set forth in Table 2 were introduced to a die and molded onto the cartridge at a temperature of approximately 199° C. and 500 psi. The cartridge was then sent to the third station where the bottom blade was retracted and the rubber guard bar is molded onto the cartridge at an elevated temperature and pressure. The blade was then repositioned and the cartridge ejected from the molding equipment.

In the first formulation, the level of MIPS was reduced from 10 wt % to 8 wt %, to accommodate the PSSNa. In the second formulation, the POLYOX WSR N-750 was reduced from 12 wt % to 10 wt %. As molded, the shaving aid strips measured approximately 3.5 centimeters long by 0.2 centimeters wide.

TABLE 2

| | Weight % | lbs |
|---|---|---|
| Formula 1 | | |
| Coagulant Polyox | 59.940 | 3.000 |
| WSR N-750 Polyox | 11.988 | 0.600 |
| PEG | 4.995 | 0.250 |
| MIPS pulverized with Teal 327 C. | 7.992 | 0.400 |
| PP pulverized with Teal 327 C. | 12.987 | 0.650 |
| PSSNa | 1.998 | 0.100 |
| Aloe | 0.100 | 0.005 |
| Total | 100.000 | 5.005 |
| Formula 2 | | |
| Coagulant Polyox | 59.940 | 3.000 |
| WSR N-750 Polyox | 9.990 | 0.500 |
| PEG | 4.995 | 0.250 |
| MIPS pulverized with Teal 327 C. | 9.990 | 0.500 |
| PP pulverized with Teal 327 C. | 12.987 | 0.650 |
| PSSNa | 1.998 | 0.100 |
| Aloe | 0.100 | 0.005 |
| Total | 100.000 | 5.005 |
| Formula 3 (Control) | | |
| Coagulant Polyox | 59.940 | 3.000 |
| WSR N-750 Polyox | 11.988 | 0.600 |
| PEG | 4.995 | 0.250 |
| MIPS pulverized with Teal 327 C. | 9.990 | 0.500 |
| PP pulverized with Teal 327 C. | 12.987 | 0.650 |
| PSSNa | — | — |
| Aloe | 0.100 | 0.005 |
| Total | 100.00 | 5.005 |

### EXAMPLE 3

This example demonstrates that the shaving aid strips of the present invention have capacity for complexing with metal ions thereby promoting the disassociation of insoluble metal salts. In twelve separate beakers, one gram of CaCO₃ was added to each beaker followed by the addition of 60 mL of deionized water. This created an excess of insoluble metal salt. The beakers were separated into four groups of three beakers. In the first group (Group I), 3 shaving heads containing the shaving aid strips made of Formula 3 (control) were added. In the second group (Group II), 3 shaving heads containing the shaving aid strips made of Formula 1 were added. In the third group (Group III), 3 shaving heads containing the shaving aid strips made of Formula 2 were added. In the fourth group (Group IV), no shaving heads were added. The beakers were allowed to equilibrate for 24 hours. Then, the contents of each beaker were filtered and the supernatant was analyzed by ICP for calcium content.

The supernatant of Group IV had an average calcium ion concentration of 8.75 μg/L. After analyses, the control (Group I) showed an average calcium ion concentration of 22.5 μg/L. Both PSSNa formulas (Group II and Group III) had average calcium ion levels of 27.4 μg/L. Subtracting out the base calcium ion level from the three sample sets, the PSSNa samples showed an increase of 35.6% in the uptake of calcium ion over the control samples, i.e.,

[Ca⁺²] average in control (Group I): 22.50 μg/L

[Ca⁺²] average in PSSNa (Group II and Group III): 27.40 μg/L

[Ca⁺²] average in water (Group IV): 8.75 μg/L

[Ca⁺²]_{GI}−[Ca⁺²]_{GIV}=22.50 μg/L−8.75 μg/L=13.75 μg/L

[Ca⁺²]_{GII/GIII}−[Ca⁺²]_{GIV}=27.40 μg/L−8.75 μg/L=18.65 μg/L

% increase={([Ca⁺²]_{GII/GIII}−[Ca⁺²]_{GI})/[Ca⁺²]_{GI}}*100=[(18.65−13.75)/13.75]*100=35.6%

Weight increase due to water absorption was also measured using 8 shaving aid strips of each formula. The average weight change of the control formulation (Formula 3) was 35.32 milligrams (“mg”). Formula 1 showed an average weight increase of 39.23 mg and Formula 2 showed an average weight increase of 40.94. Thus, in addition to showing an increase in the amount of calcium ions in the water, the water absorption property, which is indicative of lubricity, was not adversely affected.

Although the invention has been described with respect to specific aspects, those skilled in the art will recognize that others aspects are intended to be included with in the scope of the claims which follow. For example, the invention includes aspects wherein more than one shaving aid strip is provided and at least one shaving aid strip comprises the chelating agent. In addition, the invention includes an aspect wherein a shaving aid strip is provided which does not contain achelating agent, instead the chelating agent may be provided in another portion of the skin engaging portion of the shaving system. Also, it is noted that all documents referenced herein are incorporated by reference as if set out in full.

What is claimed is:

1. A shaving system comprising a blade member and a skin engaging portion in proximity to the blade member, the skin engaging portion including a chelating agent capable of reducing the amount of an insoluble metal salt in an aqueous liquid containing the insoluble metal salt.

2. The shaving system of claim 1 wherein the skin engaging portion comprises at least one of a water-sensitive polymer and a water-insoluble polymer.

3. The shaving system of claim 1 wherein the skin engaging portion includes a solid polymeric shaving aid strip comprising a water-sensitive polymer and the chelating agent.

4. The shaving system of claim 1 wherein the chelating agent is a polymer having a functional group capable of forming a chelate with a metal ion of the insoluble metal salt.

5. The shaving system of claim 1 wherein the chelating agent is a non-polymer.

6. The shaving system of claim 1 wherein the skin engaging portion has a reservoir containing a liquid, said liquid comprising the chelating agent.

7. The shaving system of claim 1 wherein the chelating agent has a decomposition temperature of at least about 150° C.

8. The shaving system of claim 1 wherein the chelating agent has a decomposition temperature of at least about 200° C.

9. The shaving system of claim 1 wherein the chelating agent has a functional group selected from the group consisting of (—SOₓ^{-y}) (Z^{+w}), (—POₓ^{-y}) (Z^{+w}), (R—COO^{-y}) (Z^{+x}), where x is 2, 3, or 4, y is less than or equal to x, R is any alkyl, aryl or aryl alkyl group, Z is any cation(s) and w is 1, 2, 3 or 4.

10. The shaving system of claim 1 wherein the chelating agent is selected from the group consisting of EDTA, calcium disodium EDTA, dipotassium EDTA, disodium EDTA, methyl cyclodextrin, pentenic acid, potassium citrate, sodium citrate, sodium gluconate, sodium metasilicate, tetrasodium EDTA, trisodium EDTA, poly(sodium 4-styrenesulfonate) and mixtures thereof.

11. The shaving system of claim 10 wherein the chelating agent is poly(sodium 4-styrenesulfonate).

12. A shaving system component comprising a polymeric element having an outer surface and an internal portion, the polymeric element comprising; (i) at least one of a water-sensitive polymer and (ii) a water-insoluble polymer, and a chelating agent capable of reducing the amount of an insoluble metal salt from an aqueous liquid containing the insoluble metal salt.

13. The shaving system component of claim 12 wherein at least one of the water-sensitive polymer or the water-insoluble polymer has a functional group capable of forming a chelate with a metal ion of the insoluble metal salt.

14. The shaving system component of claim 12 wherein the chelating agent is a polymer, different from the water-sensitive polymer and the water-insoluble polymer, having a functional group capable of forming a chelate with a metal ion of the insoluble metal salt.

15. The shaving system component of claim 12 wherein the chelating agent is provided as a coating on the outer surface of the shaving system component.

16. The shaving system component of claim 12 wherein the chelating agent is provided in the internal portion of the shaving system component.

17. The shaving system component of claim 16 wherein the chelating agent has a decomposition temperature of at least 150° C.

18. A shaving aid strip comprising a water-sensitive polymer and a chelating agent.

19. The shaving aid strip of claim 18 wherein the water-sensitive polymer is selected from the group consisting of polyvinyl compounds, polysaccharides, polyurethanes, polyacrylates, polyacrylamides, polyalkylene oxides, polysulfones, polylactones and copolymers, complexes, mixtures, and derivatives thereof.

20. The shaving aid strip of claim 18, which further comprises a water-insoluble polymer.

21. The shaving aid strip of claim 18 wherein the water-insoluble polymer is selected from the group consisting of polyethylene, polypropylene, polystyrene, butadiene-styrene copolymer, polyacetal, acrylonitrile-butadiene-styrene copolymer, ethylene vinyl acetate copolymer, polyurethanes and mixtures thereof.

22. The shaving aid strip of claim 20 wherein at least one of the water-sensitive polymer or the water-insoluble polymer has a functional group capable of forming a chelate with a metal ion of the insoluble metal salt.

23. The shaving aid strip of claim 20 wherein the chelating agent is a polymer, different from the water-sensitive polymer and the water-insoluble polymer, having a functional group capable of forming a chelate with a metal ion of the insoluble metal salt.

24. The shaving aid strip of claim 18 wherein chelating agent has a decomposition temperature of at least about 150° C.

25. The shaving aid strip of claim 18, which comprises from about 0.1 to 10 weight percent of the chelating agent based on the total weight of the shaving aid strip.

26. The shaving aid strip of claim 18 which comprises from about 0.5 to 6 weight percent of the chelating agent based on the total weight of the shaving aid strip.
